(19) United States
(12) Patent Application Publication
(10) Pub. No.: US 2020/0306176 A1
(43) Pub. Date: Oct. 1, 2020

(54) OSMOTIC PUMP CONTROLLED RELEASE FORMULATIONS

(71) Applicant: AustarPharma, LLC, Edison, NJ (US)

(72) Inventors: Rong LIU, Edison, NJ (US); Wei LI, Edison, NJ (US); Zhihong ZHANG, Edison, NJ (US)

(21) Appl. No.: 16/366,404

(22) Filed: Mar. 27, 2019

(51) Int. Cl. A61K 9/00; A61K 31/4422; A61K 47/10; A61K 47/38; A61K 47/32; A61K 31/55

(52) U.S. Cl. CPC A61K 9/0004; A61K 31/4422; A61K 31/55; A61K 47/38; A61K 47/32; A61K 47/10

(57) ABSTRACT

Disclosed is an osmotic pump controlled release formulation, comprising a core and a membrane, wherein the membrane is coated on an outer surface of the core, the formulation has at least one channel, the core is communicated with to the outer surface of the formulation via the channel, lateral length of the channel is 1.40 mm-2.50 mm, and depth of the channel is 2.05 mm-2.95 mm.

### FIELD

The present disclosure generally relates to the field of medicine. In particular, the present disclosure relates to a controlled release formulation.

### BACKGROUND

As a typical representative of sustained and controlled release formulations, an osmotic pump controlled release formulation is a drug release system with osmotic pressure difference inside and outside the coating membrane as the drug release force. The surface of an osmotic pump formulation has one or more channels for drug release. When an osmotic pump formulation is placed in a water-containing environment, water enters the interior of the coating membrane under the action of osmotic pressure difference to form a drug solution or suspension and therefore the drug is released via the channels for drug release.

### SUMMARY

In one aspect, the present disclosure relates to an osmotic pump controlled release formulation comprising a core and a membrane, wherein the membrane is coated on an outer surface of the core, the formulation has at least one channel, the core is communicated with the outer surface of the formulation via the channel, lateral length of the channel is 1.40 mm-2.50 mm, and depth of the channel is 2.05 mm-2.95 mm.

In another aspect, the present disclosure relates to a process for preparing an osmotic pump controlled release formulation, comprising:

compressing a core and forming a channel during the compression to obtain the core with at least one channel, and

coating the compressed core to obtain the osmotic pump controlled release formulation,

wherein lateral length of the channel is 1.40 mm-2.50 mm and depth of the channel is 2.05 mm-2.95 mm.

In a further aspect, the present disclosure relates to a method for improving drug release in a subject, comprising administering an osmotic pump controlled release formulation to the subject in need thereof, wherein the osmotic pump controlled release formulation comprises a core and a membrane, the membrane is coated on an outer surface of the core, the formulation has at least one channel, the core is communicated with the outer surface of the formulation via the channel, lateral length of the channel is 1.40 mm-2.50 mm, and depth of the channel is 2.05 mm-2.95 mm.

### BRIEF DESCRIPTION OF THE DRAWINGS

FIG. 1 shows the release curves of 6 parallel samples of the osmotic pump controlled release formulation A comprising nifedipine in Example 1 of the present disclosure.

FIG. 2 shows the release curves of 6 parallel samples of the osmotic pump controlled release formulation B comprising nifedipine in Example 2 of the present disclosure.

FIG. 3 shows the release curves of 6 parallel samples of the osmotic pump controlled release formulation C comprising nifedipine in Example 3 of the present disclosure.

FIG. 4 shows the release curves of 6 parallel samples of the osmotic pump controlled release formulation D comprising nifedipine in Example 4 of the present disclosure.

FIG. 5 shows the release curves of 6 parallel samples of the osmotic pump controlled release formulation E comprising nifedipine in Example 5 of the present disclosure.

FIG. 6 shows the release curves of 6 parallel samples of the osmotic pump controlled release formulation F comprising nifedipine in Example 6 of the present disclosure.

FIG. 7 shows the release curves of 6 parallel samples of the osmotic pump controlled release formulation G comprising carbamazepine in Example 7 of the present disclosure.

FIG. 8 shows a schematic view of a longitudinal section of an osmotic pump controlled release formulation in one embodiment of the present disclosure.

FIG. 9 shows a schematic view of a longitudinal section of an osmotic pump controlled release formulation in another embodiment of the present disclosure.

FIG. 10 shows a schematic view of a longitudinal section of an osmotic pump controlled release formulation in a further embodiment of the present disclosure.

### DETAILED DESCRIPTION

In the following description, certain specific details are included to provide a thorough understanding to various disclosed embodiments. However, one skilled in the art will recognize that embodiments may be practiced without one or more of these specific details, but with other methods, components, materials, etc.

Unless otherwise required in the present disclosure, throughout the specification and the attached claims, the words “comprise”, “include”, and “have” shall be interpreted in an open, inclusive sense, i.e., “including but not limited to”.

“One embodiment”, “an embodiment”, “in another embodiment” or “in some embodiments” as referred to throughout the specification means that specific reference elements, structures, or features described in connection with the embodiment are included in at least one embodiment. Therefore, the phrases “in one embodiment” or “in an embodiment” or “in another embodiment” or “in some embodiments” appearing at different locations throughout the specification are not necessarily all referring to the same embodiment. Furthermore, specific elements, structures, or features may be combined in any suitable manner in one or more embodiments.

### Definitions

As used herein, the term “lateral length of the channel” refers to the maximum radial dimension of the channel formed in the core in the process of compressing the core.

As used herein, the term “depth of the channel” refers to the distance from the opening to the bottom of the channel formed in the core in the process of compressing the core.

As used herein, the term “pharmaceutically acceptable salt” includes “acceptable acid addition salt” and “acceptable base addition salt”.

As used herein, the term “acceptable acid addition salt” refers to those salts that maintain the biological effectiveness and properties of free bases, and the acid addition salts are biologically or otherwise suitable and are formed by inorganic acids or organic acids. The inorganic acids such as, but not limited to, hydrochloric acid, hydrobromic acid, sulfuric acid, nitric acid, phosphoric acid, etc., and the organic acids such as, but not limited to, acetic acid, 2,2-dichloroacetic acid, adipic acid, alginic acid, ascorbic acid, aspartic acid, benzene sulfonic acid, benzene carboxylic acid, 4-acetamido benzene carboxylic acid, camphoric acid, camphor-10-sulfonic acid, capric acid, caproic acid, caprylic acid, carbonic acid, cinnamic acid, citric acid, cyclohexylaminosulfonic acid, dodecyl sulfuric acid, ethane-1,2-disulfonic acid, ethanesulfonic acid, 2-hydroxy ethanesulfonic acid, formic acid, fumaric acid, muconic acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, galacturonic acid, lauric acid, maleic acid, malic acid, malonic acid, mandelic acid, methanesulfonic acid, muconic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, dihydroxynaphthalene acid, propionic acid, pyroglutamic acid, pyruvic acid, salicylic acid, 4-aminosalicylic acid, sebacic acid, stearic acid, succinic acid, tartaric acid, thiocyanate, p-toluenesulfonic acid, trifluoroacetic acid, undecylenic acid, and the like.

As used herein, the term “acceptable base addition salt” refers to those salts that maintain the biological effectiveness and properties of free acids and the base addition salts are biologically or otherwise suitable. Inorganic or organic bases are added to the free acid to prepare these salts. Salts derived from inorganic base include, but not limited to, sodium, potassium, lithium, ammonium, calcium, magnesium, iron, zinc, copper, manganese, aluminum salts and the like. In some embodiments, the inorganic salts are ammonium, sodium, potassium, calcium and magnesium salts. Salts derived from organic bases include but not limited to salts of primary, secondary and tertiary amines, substituted amines including natural substituted amines, cyclic amines and salts of basic ion exchange resins, such as, ammonia, isopropylamine, trimethylamine, diethylamine, triethylamine, tripropylamine, diethanolamine, ethanolamine, 2-dimethylaminoethanol, 2-diethylaminoethanol, dicyclohexylamine, lysine, arginine, histidine, caffeine, procaine, hypamine, choline, betaine, benzylamine, phenylethylenediamine, ethylenediamine, glucosamine, methylglucosamine, theobromine, triethanolamine, aminobutyrol, purine, piperazine, piperidine, N-ethylpiperidine, polyamine resin, etc. In some embodiments, the organic bases are isopropylamine, diethylamine, ethanolamine, trimethylamine, dicyclohexylamine, choline and caffeine.

As used herein, the term “active pharmaceutical ingredient” refers to a chemical entity that can effectively treat a target disorder, disease or condition.

As used herein, the term “pharmaceutically acceptable excipients” refers to excipients that can be used in the pharmaceutical field and harmless to products or mammals, or have a reasonable or acceptable benefit/risk ratio. Pharmaceutically acceptable excipients include, but not limited to: a) fillers/diluents, such as starch, compressible starch, powdered sugar, dextrin, lactose, microcrystalline cellulose, inorganic salts, sugar alcohols, etc.; b) wetters/binders, such as water, ethanol, starch slurry, dextrin, sugar powder and syrup, mucilage, polyethylene glycol 4000, cellulose derivatives, methyl cellulose, hydroxypropyl cellulose, hydroxypropyl methyl cellulose, sodium hydroxymethyl cellulose, ethyl cellulose, etc.; c) disintegrants, such as dry starch, sodium hydroxymethyl starch, low-substituted hydroxypropyl cellulose, cross-linked polyvinylpyrrolidone, cross-linked sodium carboxymethyl cellulose, effervescent disintegrants, etc.; d) surfactants, such as Arabic gum, tragacanth gum, gelatin, propylene glycol monostearate, glyceryl monostearate, ethylene distearate, diglyceride monooleate, sodium dodecyl sulfate, Span 20, Span 40, Span 60, Span 65, Span 80, Span 83, Span 85, potassium oleate, sodium oleate, triethanolamine oleate, lecithin, sucrose ester, poloxamer 188, Atlas G-263, Tween 20, Tween 21, Tween 40, Tween 60, Tween 61, Tween 65, Tween 80, Tween 81, Tween 85, Myrj 45, Myrj 49, Myrj 51, Myrj 52, polyoxyethylene 400 monolaurate, polyoxyethylene 400 monostearate, polyoxyethylene 400 monooleate, Brij 35, Brij 30, cetomacrogol, polyoxyethylene hydrogenated castor oil, polyoxyethylene alkyl phenol, polyoxyethylene nonyl phenol ether, etc.; e) lubricants, such as magnesium stearate, stearic acid, glyceryl behenate, micro powder silica gel, talc, hydrogenated vegetable oil, polyethylene glycol, magnesium lauryl sulfate, etc.; f) colorants, such as amaranth red, carmine, new red, iron oxide red, lemon yellow, sunset yellow, indigo, brilliant blue, beet red, lac red, bilberry red, capsanthin, red kermel color, etc.; g) flavoring agents, such as sweeteners, fragrances, mucilages, effervescing agents, etc.; h) antioxidants or preservatives, such as tert-butyl hydroxy anisole, dibutyl hydroxy toluene, etc.; i) osmotic promoting polymers, such as polyoxyethylene, polyvinylpyrrolidone, polyacrylic acid, hydroxypropyl methyl cellulose, polyhydroxyalkyl methacrylate, etc.; and j) osmotic promoters, such as sodium chloride, magnesium chloride, magnesium sulfate, sodium sulfate, potassium sulfate, tartaric acid, lactose, mannitol, urea, etc.

As used herein, the term “increase of coating weight” refers to the ratio of the weight of the membrane to the weight of the core before coating.

As used herein, the term “drug-containing layer” refers to a core comprising an active pharmaceutical ingredient in an osmotic pump formulation. In an aqueous environment, water enters the drug-containing layer under the action of osmotic pressure difference to form a solution or suspension of an active pharmaceutical ingredient, which can be released via channels.

As used herein, the term “push layer” refers to a swellable core comprising an active osmotic promoting substance in an osmotic pump formulation. In an aqueous environment, the push layer absorbs water and swells, pushes the solution or suspension of an active pharmaceutical ingredient in the drug-containing layer to be released via channels.

As used herein, the term “in-situ pore-forming technology” refers to the technology of forming a channel while compressing before coating, and then coating semi-permeable membrane to form an osmotic pump formulation.

### DETAILED DESCRIPTION

In one aspect, the present disclosure relates to an osmotic pump controlled release formulation comprising a core and a membrane, wherein the membrane is coated on an outer surface of the core, the formulation has at least one channel, the core is communicated with the outer surface of the formulation via the channel, lateral length of the channel is about 1.40 mm-2.50 mm, and depth of the channel is about 2.05 mm-2.95 mm.

In some embodiments, the lateral length of the channel is about 1.70 mm-2.20 mm.

In some embodiments, the lateral length of the channel is about 1.40 mm.

In some embodiments, the lateral length of the channel is about 1.45 mm.

In some embodiments, the lateral length of the channel is about 1.50 mm.

In some embodiments, the lateral length of the channel is about 1.55 mm.

In some embodiments, the lateral length of the channel is about 1.60 mm.

In some embodiments, the lateral length of the channel is about 1.65 mm.

In some embodiments, the lateral length of the channel is about 1.70 mm.

In some embodiments, the lateral length of the channel is about 1.75 mm.

In some embodiments, the lateral length of the channel is about 1.80 mm.

In some embodiments, the lateral length of the channel is about 1.85 mm.

In some embodiments, the lateral length of the channel is about 1.90 mm.

In some embodiments, the lateral length of the channel is about 1.95 mm.

In some embodiments, the lateral length of the channel is about 2.00 mm.

In some embodiments, the lateral length of the channel is about 2.05 mm.

In some embodiments, the lateral length of the channel is about 2.10 mm.

In some embodiments, the lateral length of the channel is about 2.15 mm.

In some embodiments, the lateral length of the channel is about 2.20 mm.

In some embodiments, the lateral length of the channel is about 2.25 mm.

In some embodiments, the lateral length of the channel is about 2.30 mm.

In some embodiments, the lateral length of the channel is about 2.35 mm.

In some embodiments, the lateral length of the channel is about 2.40 mm.

In some embodiments, the lateral length of the channel is about 2.45 mm.

In some embodiments, the lateral length of the channel is about 2.50 mm.

In some embodiments, the depth of the channel is about 2.25 mm-2.75 mm.

In some embodiments, the depth of the channel is about 2.05 mm.

In some embodiments, the depth of the channel is about 2.10 mm.

In some embodiments, the depth of the channel is about 2.15 mm.

In some embodiments, the depth of the channel is about 2.20 mm.

In some embodiments, the depth of the channel is about 2.25 mm.

In some embodiments, the depth of the channel is about 2.30 mm.

In some embodiments, the depth of the channel is about 2.35 mm.

In some embodiments, the depth of the channel is about 2.40 mm.

In some embodiments, the depth of the channel is about 2.45 mm.

In some embodiments, the depth of the channel is about 2.50 mm.

In some embodiments, the depth of the channel is about 2.55 mm.

In some embodiments, the depth of the channel is about 2.60 mm.

In some embodiments, the depth of the channel is about 2.65 mm.

In some embodiments, the depth of the channel is about 2.70 mm.

In some embodiments, the depth of the channel is about 2.75 mm.

In some embodiments, the depth of the channel is about 2.80 mm.

In some embodiments, the depth of the channel is about 2.85 mm.

In some embodiments, the depth of the channel is about 2.90 mm.

In some embodiments, the depth of the channel is about 2.95 mm.

In some embodiments, exemplary examples of bottom shapes that can be used in the channels of the present disclosure include, but not limited to circle, oval, square, rectangle, diamond and triangle.

In some embodiments, the core is single-layered or multi-layered.

In some embodiments, the multi-layered core is double-layered.

In some embodiments, the multi-layered core is three-layered.

In some embodiments, the multi-layered core is four-layered.

In some embodiments, the appropriate number of layers can be selected according to actual needs.

In some embodiments, the core comprises at least one drug-containing layer.

In some embodiments, the core is single-layered, wherein the single-layered is a drug-containing layer.

In some embodiments, the core is double-layered, wherein the first layer of the double layers is a drug-containing layer, and the second layer is a push layer.

In some embodiments, the core is three-layered, wherein the first layer of the three layers is a drug-containing layer, the second layer is a drug-containing layer, and the third layer is a push layer.

In some embodiments, the core is four-layered, wherein at least one of the four layers is a drug-containing layer.

In some embodiments, the drug-containing layer comprises an active pharmaceutical ingredient, an osmotic promoting polymer, and optionally other pharmaceutically acceptable excipients.

In some embodiments, the drug-containing layer comprises an active pharmaceutical ingredient, an osmotic promoting polymer, a lubricant and optionally other pharmaceutically acceptable excipients.

In some embodiments, the push layer comprises an osmotic promoting polymer, an osmotic promoting agent and optionally other pharmaceutically acceptable excipients.

In some embodiments, the push layer comprises an osmotic promoting polymer, an osmotic promoting agent, a lubricant and optionally other pharmaceutically acceptable excipients.

In some embodiments, exemplary examples of active pharmaceutical ingredients that can be used in the present disclosure include, but not limited to, antihypertensive agents, hypolipidemic agents, oral antidiabetic agents, non-steroidal anti-inflammatory agents, β-receptor antagonists, calcium ionic channel blockers, serotonin reuptake inhibitors, antipsychotic agents and antibacterial agents.

In some embodiments, exemplary examples of antihypertensive agents that can be used in the present disclosure include, but not limited to, sympathetic nerve agents, such as reserpine, deserpidine, methoserpidine, mecamylamine and pempidine; vasodilator agents, such as hydralazine, dihydralazine, todralazine, budralazine, minoxidil, pinacidil and sodium nitroprusside; agents affecting renin-angiotensin-aldosterone system, such as enalkiren, remikiren, aliskiren, teprotide, captopril, enalapril maleate and losartan; and calcium ionic channel blockers, such as nifedipine, nicardipine hydrochloride, nitrendipine, felodipine, isradipine, amlodipine besylate, cilnidipine, lacidipine, clevidipine, diltiazem, verapamil hydrochloride and bepridil.

In some embodiments, exemplary examples of hypolipidemic agents that can be used in the present disclosure include, but not limited to, nicotinic acid, olbemox, inositol nicotinate, clofibrate, lovastatin, pravastatin, simvastatin, atorvastatin, probucol, cholestyramine, colestipol, divistyramine, fenofibrate, pantethine, linoleic acid and ω-3 fatty acids.

In some embodiments, exemplary examples of oral antidiabetic agents that can be used in the present disclosure include, but not limited to, insulin and analogues thereof, such as regular insulin, insulin aspartate, insulin lispro and insulin glargine; insulin secretion promoters, such as glybutamide, tolbutamide, glipizide, glimepiride and repaglinide; insulin sensitizers, such as metformin hydrochloride; α-glucosidase inhibitors, such as voglibose and miglitol; dipeptidyl peptidase-4-inhibitors, such as sitagliptin phosphate.

In some embodiments, exemplary examples of non-steroidal anti-inflammatory agents that can be used in the present disclosure include, but not limited to, indomethacin, diclofenac sodium, ibuprofen, naproxen, pirooxcam, meloxicam, celecoxib and imrecoxib.

In some embodiments, exemplary examples of β-receptor antagonists that can be used in the present disclosure include, but not limited to, propranolol, pindolol, timolol, metoprolol, atenolol, betaxolol, bisoprolol, labetalol, carvedilol and arotinolol.

In some embodiments, exemplary examples of calcium ionic channel blockers that can be used in the present disclosure include, but not limited to, nifedipine, nicardipine hydrochloride, nitrendipine, felodipine, isradipine, amlodipine besylate, cilnidipine, lacidipine, clevidipine, diltiazem, verapamil hydrochloride and bepridil.

In some embodiments, exemplary examples of serotonin reuptake inhibitors that can be used in the present disclosure include, but not limited to, fluoxetine, sertraline, paroxetine, citalopram and fluvoxamine.

In some embodiments, exemplary examples of antipsychotic agents that can be used in the present disclosure include, but not limited to, chlorpromazine hydrochloride, perphenazine, fluphenazine decanoate, chlorprothixene, haloperidol and sulpiride.

In some embodiments, exemplary examples of antibacterial agents that can be used in the present disclosure include, but not limited to, synthetic antibacterial agents such as sulfamethoxazole, trimethoprim, norfloxacin, ciprofloxacin, levofloxacin and linezolid; and antibiotic agents such as penicillin, cephalosporin C, clavulanic acid and sulbactam.

In some embodiments, illustrative examples of active pharmaceutical ingredients that can be used in the present disclosure include, but not limited to, prazosin hydrochloride, phenylpropanolamine hydrochloride, doxazosin mesylate, verapamil hydrochloride, oxybutynin hydrochloride, isradipine, paliperidone, chlorpheniramine maleate, glipizide, nifedipine, pseudoephedrine hydrochloride, pseudoephedrine sulfate, aluminum sulfate, carbamazepine, leuprorelin acetate, sufentanil citrate and methylphenidate hydrochloride.

In some embodiments, the active pharmaceutical ingredient is nifedipine.

In some embodiments, the active pharmaceutical ingredient is carbamazepine.

In some embodiments, exemplary examples of osmotic promoting polymers that can be used in the present disclosure include, but not limited to, polyoxyethylene, polyvinylpyrrolidone, polyacrylic acid, hydroxypropyl methylcellulose, polyhydroxyalkyl methacrylate and a mixture thereof.

In some embodiments, exemplary examples of osmotic promoters that can be used in the present disclosure include, but not limited to, sodium chloride, magnesium chloride, magnesium sulfate, sodium sulfate, potassium sulfate, tartaric acid, lactose, mannitol, urea and a mixture thereof.

In some embodiments, exemplary examples of lubricants that can be used in the present disclosure include, but not limited to, magnesium stearate, stearic acid, glyceryl behenate and a mixture thereof.

In some embodiments, exemplary examples of membranes that can be used in the present disclosure include, but not limited to, semi-permeable membrane materials.

In some embodiments, exemplary examples of semi-permeable membrane materials that can be used in the present disclosure include, but not limited to, cellulose acetate, cellulose acetate aqueous dispersion, ethyl cellulose, polyvinyl chloride, polycarbonate, vinyl alcohol-vinyl acetate, ethylene-propylene polymer, polyvinyl acetate, polyvinyl acetate/polyvinyl pyrrolidone composites and a mixture thereof.

In some embodiments, the membranes comprise pore-forming agents.

In some embodiments, exemplary examples of pore-forming agents that can be used in the present disclosure include, but not limited to, glycerol, sorbitol, polyethylene glycol 400, polyethylene glycol 600, polyethylene glycol 1000, polyethylene glycol 1500, polyethylene glycol 4000, hydroxypropyl methyl cellulose, polyvinyl alcohol and a mixture thereof.

In some embodiments, when the drug release channel of the osmotic pump controlled release formulation prepared by the in-situ pore-forming technology have a specific range of lateral length and the depth, the drug release of the osmotic pump controlled release formulation is smoother and is more consistent regarding the initial release time.

In some embodiments, when the lateral length of the preformed drug release channel exceeds 2.50 mm or is less than 1.40 mm, the degree of coverage of the inner wall of the drug release channel is difficult to be stable and homogeneous during the coating process.

In some embodiments, when the depth of the preformed drug release channel is less than 2.05 mm, the degree of coverage of the inner wall of the drug release channel is difficult to be stable and homogeneous during the coating process.

In some embodiments, although the extra depth of the preformed drug release channel does not lead to heterogeneous coverage of the inner wall, the protruding portion of a punch can be damaged during compression.

In some embodiments, when more than 5% of the inner wall of the drug release channel is covered by the semi-permeable membrane material, the lag time of drug release of an osmotic pump tablet becomes longer.

In some embodiments, when more than 10% of the inner wall of the drug release channel is covered by the semi-permeable membrane material, the lag time of drug release of an osmotic pump tablet becomes longer.

In some embodiments, when more than 15% of the inner wall of the drug release channel is covered by the semi-permeable membrane material, the lag time of drug release of an osmotic pump tablet becomes longer.

In some embodiments, when more than 20% of the inner wall of the drug release channel is covered by the semi-permeable membrane material, the lag time of drug release of an osmotic pump tablet becomes longer.

In some embodiments, when more than 25% of the inner wall of the drug release channel is covered by the semi-permeable membrane material, the lag time of drug release of an osmotic pump tablet becomes longer.

In some embodiments, when more than 30% of the inner wall of the drug release channel is covered by the semi-permeable membrane material, the lag time of drug release of an osmotic pump tablet becomes longer.

In some embodiments, when more than 35% of the inner wall of the drug release channel is covered by the semi-permeable membrane material, the lag time of drug release of an osmotic pump tablet becomes longer.

In some embodiments, when more than 40% of the inner wall of the drug release channel is covered by the semi-permeable membrane material, the lag time of drug release of an osmotic pump tablet becomes longer.

In some embodiments, when more than 45% of the inner wall of the drug release channel is covered by the semi-permeable membrane material, the lag time of drug release of an osmotic pump tablet becomes longer.

In some embodiments, when more than 50% of the inner wall of the drug release channel is covered by the semi-permeable membrane material, the lag time of drug release of an osmotic pump tablet becomes longer.

In some embodiments, when more than 55% of the inner wall of the drug release channel is covered by the semi-permeable membrane material, the lag time of drug release of an osmotic pump tablet becomes longer.

In some embodiments, when more than 60% of the inner wall of the drug release channel is covered by the semi-permeable membrane material, the lag time of drug release of an osmotic pump tablet becomes longer.

In some embodiments, when more than 65% of the inner wall of the drug release channel is covered by the semi-permeable membrane material, the lag time of drug release of an osmotic pump tablet becomes longer.

In some embodiments, when more than 70% of the inner wall of the drug release channel is covered by the semi-permeable membrane material, the lag time of drug release of an osmotic pump tablet becomes longer.

In some embodiments, when more than 75% of the inner wall of the drug release channel is covered by the semi-permeable membrane material, the lag time of drug release of an osmotic pump tablet becomes longer.

In some embodiments, when more than 80% of the inner wall of the drug release channel is covered by the semi-permeable membrane material, the lag time of drug release of an osmotic pump tablet becomes longer.

In some embodiments, when more than 85% of the inner wall of the drug release channel is covered by the semi-permeable membrane material, the lag time of drug release of an osmotic pump tablet becomes longer.

In some embodiments, when more than 90% of the inner wall of the drug release channel is covered by the semi-permeable membrane material, the lag time of drug release of an osmotic pump tablet becomes longer.

In some embodiments, when more than 95% of the inner wall of the drug release channel is covered by the semi-permeable membrane material, the lag time of drug release of an osmotic pump tablet becomes longer.

In some embodiments, when more than 100% of the inner wall of the drug release channel is covered by semi-permeable membrane material, the lag time of drug release of an osmotic pump tablet becomes longer.

In some embodiments, the coverage degree of the channel is not the only reason that affects the lag time of the drug release. The coverage degree of the channel together with the composition and proportion of other components of an osmotic pump tablet constitutes the complicated factors of influence.

In some embodiments, the osmotic pump formulation absorbs water through osmotic substances in the core, causing the propellant to expand and pushing the drug to release. In general, osmotic pump formulations have a release lag time of about one to two hours and the active pharmaceutical ingredients generally are released after two hours. Therefore, it is very important to have a stable and homogeneous coating area in the channel of the osmotic pump formulation, because once the degree of coverage of the channel covered by the semi-permeable membrane material is heterogeneous and the degree of non-coverage of the inner wall of the channel is different, it is difficult for osmotic pump formulations to obtain a consistent initial release time. When the non-coverage area of the inner wall of the channel is small, the osmotic pump formulations always tend to have a longer release lag time, resulting in an extension of the initial release time of the active pharmaceutical ingredients, which affects the initial release and in vivo absorption of the active pharmaceutical ingredients, especially the active pharmaceutical ingredients that can be rapidly absorbed in vivo.

As it is generally believed that osmotic pump formulations have a release lag time of one to two hours, the release behavior of osmotic pump formulations within a certain time after the lag time is a key parameter for evaluating the stability of drug release behavior of osmotic pump formulations. Therefore, the dispersion degree of the release amount of the osmotic pump controlled release formulation in four hours is used to evaluate whether the release initial time of osmotic pump tablets is delayed or acceptable. If the osmotic pump tablets have a relatively consistent initial release time, the release amounts thereof in four hours have a smaller dispersion degree, i.e. smaller SD (standard deviation) values.

In another aspect, the present disclosure relates to a process for preparing an osmotic pump controlled release formulation, comprising:

compressing a core and forming a channel during the compression to obtain the core having at least one channel, and

coating the compressed core to obtain the osmotic pump controlled release formulation,

wherein lateral length of the channel is about 1.40 mm-2.50 mm and depth of the channel is about 2.05 mm-2.95 mm.

In some embodiments, the increase of coating weight is about 1.00%-20.00%.

In some embodiments, the increase of coating weight gain is about 2.00%-18.00%.

In some embodiments, the increase of coating weight gain is about 3.00%-16.00%.

In a further aspect, the present disclosure relates to a method for improving drug release in a subject, comprising administering an osmotic pump controlled release formulation to a subject in need thereof, wherein the osmotic pump controlled release formulation comprises a core and a membrane, the membrane is coated on an outer surface of the core, the formulation has at least one channel, the core is communicated with the outer surface of the formulation via the channel, lateral length of the channel is about 1.40 mm-2.50 mm, and depth of the channel is about 2.05 mm-2.95 mm.

In some embodiments, the osmotic pump controlled release formulation is administered twice a day.

In some embodiments, the osmotic pump controlled release formulation is administered once a day.

In some embodiments, the osmotic pump controlled release formulation is administered once every 2 days.

Hereinafter, the present disclosure will be explained in detail by the following examples in order to better understand various aspects of the present disclosure and its advantages. However, it would be appreciated that the following examples are non-limiting and are only intended to illustrate some embodiments of the present disclosure.

### EXAMPLES

### Example 1: Osmotic Pump Controlled Release Formulation A Comprising Nifedipine

The composition of the formulation A was as follows:

| | | mg/tablet | g/200 tablets | wt % |
|---|---|---|---|---|
| drug- | nifedipine | 99 | 19.8 | 13.20 |
| containing | polyoxyethylene | 328 | 65.6 | 43.73 |
| layer | N80 | | | |
| | sodium chloride | 47 | 9.4 | 6.27 |
| | magnesium stearate | 1 | 0.2 | 0.13 |
| push layer | polyoxyethylene | 213 | 42.6 | 28.40 |
| | WSR301 | | | |
| | sodium chloride | 60 | 12. | 8.00 |
| | iron oxide red | 1 | 0.2 | 0.13 |
| | magnesium stearate | 1 | 0.2 | 0.13 |

Manufacturer/log number information of active pharmaceutical ingredients and excipients:

Nifedipine: Moehs/17045001

Polyoxyethylene N80: Dow Chemical Company/14204001

Sodium chloride: AVANTOR MATERIALS/15077001

Magnesium stearate: MOLLINCKRODT/16347001

Polyoxyethylene WSR301: Dow Chemical Company/15296001

Iron oxide red: Huntsman Pigments and Additives/14196001

Cellulose acetate: Eastman Chemical LTD./29360

Acetone: Guangzhou Chemical Reagent Factory/20171001-2

Equipment information:

Manual tablet press: CARVER/3850-7

High-efficiency coating machine: Zhejiang Xiaolun Pharmaceutical Machinery Co., Ltd/BGB-5F

Off-line automated sampling dissolution tester: SOTAX/AT xtend

High Performance Liquid Chromatography: Shimadzu/LC-20AT

The preparation process was as follows:

a) Preparation of the drug-containing layer mixture: Nifedipine, sodium chloride and polyoxyethylene N80 were mixed according to the composition in the above table. The mixture was sieved with a 20-mesh sieve to crush lumps. Finally, magnesium stearate was added and mixed homogeneously to obtain the mixture M. The mixture M was compressed into tablets with a punch having a diameter of 12 mm. The tablets were crushed in a mortar and sieved with a 20-mesh sieve to obtain the granular drug-containing layer mixture N.

b) Preparation of the push layer mixture: According to the above composition, polyoxyethylene WSR301, sodium chloride and iron oxide red were mixed homogeneously. Magnesium stearate was added into the mixture to obtain the powdery push layer mixture 0.

c) Preparation of the core with channel: A punch with 12 mm diameter was used and there was a cylindrical protrusion on the surface of the punch. Firstly, the push layer mixture 0 was compressed to give a push layer. The drug-containing layer mixture N was compressed to give a drug-containing layer on the push layer and therefore a double-layered core with channel was obtained. The lateral length of the formed channel was 1.95 mm and the depth of the formed channel was 2.00 mm.

| | | Amount (g) |
|---|---|---|
| coating layer | cellulose acetate | 144 |
| | acetone | 4000 |

d) Film coating: Cellulose acetate was added into acetone. The mixture was stirred until cellulose acetate was dissolved to obtain a coating solution. The cores were placed in a coating machine and coated with the coating solution. The increase of coating weight was 3.47%.

The osmotic pump controlled release formulation A comprising nifedipine prepared according to the composition had one channel. The drug-containing layer of the core was communication with the outer surface of the osmotic pump controlled release formulation A comprising nifedipine via the channel. The lateral length of the channel was 1.95 mm and the depth of the channel was 2.00 mm.

The dissolution of the osmotic pump controlled release formulation A comprising nifedipine was determined with the Apparatus II (Paddle) method specified in the United States Pharmacopoeia (USP), in which the speed of rotation was 100 rpm, the temperature of water bath was (37.0±0.5°) C., and the dissolution medium was 900 mL phosphate buffer solution containing 1% sodium dodecyl sulfate (SLS) with pH=6.8. The dissolution test was carried out with dissolution tester and Agilent HPLC. 6 parallel samples (Tablets 1-6) of the osmotic pump controlled release formulation A comprising nifedipine were put into dissolution cups, respectively. 5 mL of solutions were sampled at 2 h, 4 h, 8 h, 12 h, 16 h, 20 h and 24 h, respectively. The solutions were filtered with 0.45 μm microporous filter membrane, while 5 mL of dissolution medium with the same temperature was added. The subsequent filtrate was subject to HPLC.

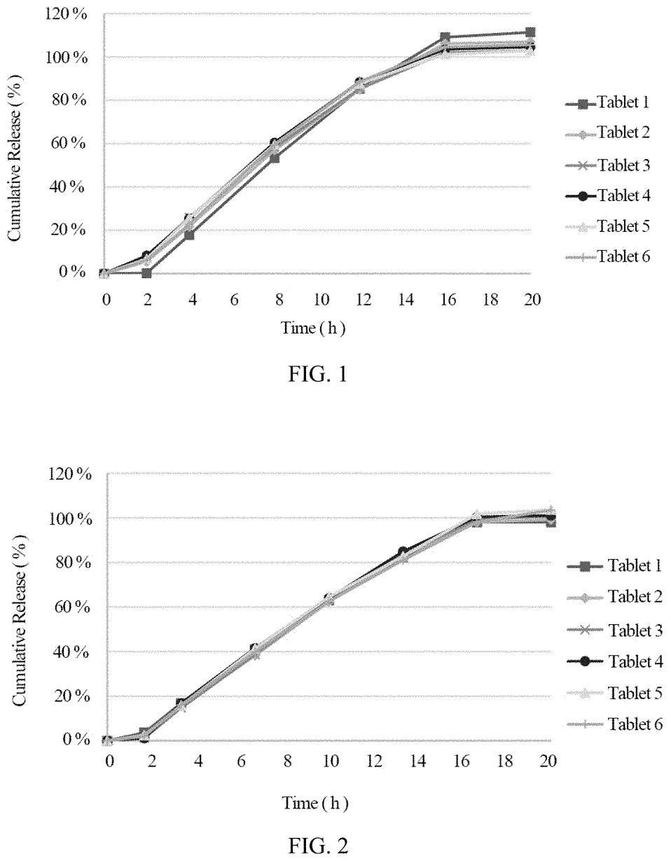

The cumulative release of the osmotic pump controlled release formulation A comprising nifedipine (Tablets 1-6) at different dissolution times were calculated, respectively, as shown in Table 1. The cumulative dissolution curves were plotted as shown in FIG. 1.

TABLE 1
Cumulative release of the osmotic pump controlled release formulation A comprising nifedipine (n = 6)

| Time | Cumulative Release (%) | | | | | | | |
|---|---|---|---|---|---|---|---|---|
| (h) | Tablet 1 | Tablet 2 | Tablet 3 | Tablet 4 | Tablet 5 | Tablet 6 | Mean | SD |
| 0 | 0.0 | 0.0 | 0.0 | 0.0 | 0.0 | 0.0 | 0 | 0.000 |
| 2 | 0.2 | 5.5 | 7.7 | 8.3 | 6.8 | 6.2 | 5.8 | 2.914 |
| 4 | 17.8 | 21.8 | 26.0 | 25.5 | 25.7 | 22.7 | 23.3 | 3.185 |
| 8 | 53.3 | 57.0 | 58.3 | 60.5 | 59.6 | 59.4 | 58.0 | 2.606 |
| 12 | 85.3 | 84.9 | 85.3 | 88.5 | 87.7 | 88.6 | 86.7 | 1.733 |
| 16 | 109.1 | 106.2 | 102.0 | 103.5 | 101.2 | 104.6 | 104.4 | 2.904 |
| 20 | 111.4 | 107.0 | 102.9 | 104.6 | 102.9 | 105.6 | 105.7 | 3.196 |

### Example 2: Osmotic Pump Controlled Release Formulation B Comprising Nifedipine

The composition of the formulation B was as follows:

| | | mg/tablet | g/200 tablets | wt % |
|---|---|---|---|---|
| drug- | nifedipine | 99 | 19.8 | 13.20 |
| containing | polyoxyethylene | 328 | 65.6 | 43.73 |
| layer | N80 | | | |
| | sodium chloride | 47 | 9.4 | 6.27 |
| | magnesium stearate | 1 | 0.2 | 0.13 |
| push layer | polyoxyethylene | 213 | 42.6 | 28.40 |
| | WSR301 | | | |
| | sodium chloride | 60 | 12 | 8.00 |
| | iron oxide red | 1 | 0.2 | 0.13 |
| | magnesium stearate | 1 | 0.2 | 0.13 |

Manufacturer/log number information of active pharmaceutical ingredients and excipients:

Nifedipine: Moehs/17045001

Polyoxyethylene N80: Dow Chemical Company/14204001

Sodium chloride: AVANTOR MATERIALS/15077001

Magnesium stearate: MOLLINCKRODT/16347001

Polyoxyethylene WSR301: Dow Chemical Company/15296001

Iron oxide red: Huntsman Pigments and Additives/14196001

Cellulose acetate: Eastman Chemical LTD./29360

Acetone: Guangzhou Chemical Reagent Factory/20171001-2

Equipment information:

Manual tablet press: CARVER/3850-7

High-efficiency coating machine: Zhejiang Xiaolun Pharmaceutical Machinery Co., Ltd/BGB-5F

Off-line automated sampling dissolution tester: SOTAX/AT xtend

High Performance Liquid Chromatography: Shimadzu/LC-20AT

The preparation process was as follows:

a) Preparation of the drug-containing layer mixture: Nifedipine, sodium chloride and polyoxyethylene N80 were mixed according to the composition in the above table. The mixture was sieved with a 20-mesh sieve to crush lumps. Finally, magnesium stearate was added and mixed homogeneously to obtain the mixture M. The mixture M was compressed into tablets with a punch having a diameter of 12 mm. The tablets were crushed in a mortar and sieved with a 20-mesh sieve to obtain the granular drug-containing layer mixture N.

b) Preparation of the push layer mixture: According to the above composition, polyoxyethylene WSR301, sodium chloride and iron oxide red were mixed homogeneously. Magnesium stearate was added into the mixture to obtain the powdery push layer mixture 0.

c) Preparation of the core with channel: A punch with 12 mm diameter was used and there was a cylindrical protrusion on the surface of the punch. Firstly, the push layer mixture 0 was compressed to give a push layer. The drug-containing layer mixture N was compressed to give a drug-containing layer on the push layer and therefore a double-layered core with channel was obtained. The lateral length of the formed channel was 1.56 mm and the depth of the formed channel was 2.5 mm.

| | | Amount (g) |
|---|---|---|
| coating layer | cellulose acetate | 144 |
| | acetone | 4000 |

d) Film coating: Cellulose acetate was added into acetone. The mixture was stirred until cellulose acetate was dissolved to obtain a coating solution. The cores were placed in a coating machine and coated with the coating solution. The increase of coating weight was 5.33%.

The osmotic pump controlled release formulation B comprising nifedipine prepared according to the composition had one channel. The drug-containing layer of the core was communicated with the outer surface of the osmotic pump controlled release formulation B comprising nifedipine via the channel. The lateral length of the channel was 1.56 mm and the depth of the channel was 2.50 mm.

The dissolution of the osmotic pump controlled release formulation B comprising nifedipine was determined with the Apparatus II (Paddle) method specified in the United States Pharmacopoeia (USP), in which the speed of rotation was 100 rpm, the temperature of water bath was (37.0±0.5°) C., and the dissolution medium was 900 mL phosphate buffer solution of containing 1% sodium dodecyl sulfate (SLS) with pH=6.8. The dissolution test was carried out with dissolution tester and Agilent HPLC. 6 parallel samples (Tablets 1-6) of the osmotic pump controlled release formulation B comprising nifedipine were put into dissolution cups, respectively. 5 mL of solutions were sampled at 2 h, 4 h, 8 h, 12 h, 16 h, 20 h and 24 h, respectively. The samples were filtered with 0.45 μm microporous filter membrane, while 5 mL of dissolution medium with the same temperature was added. The subsequent filtrate was subject to HPLC. The cumulative release of the osmotic pump controlled release formulation B comprising nifedipine (Tablets 1-6) at different dissolution times were calculated, respectively, as shown in Table 2. The cumulative dissolution curves were plotted as shown in FIG. 2.

TABLE 2
Cumulative release of the osmotic pump controlled release formulation B comprising nifedipine (n = 6)

| Time | Cumulative Release (%) | | | | | | | |
|---|---|---|---|---|---|---|---|---|
| (h) | Tablet 1 | Tablet 2 | Tablet 3 | Tablet 4 | Tablet 5 | Tablet 6 | Mean | SD |
| 0 | 0 | 0 | 0 | 0 | 0 | 0 | 0 | 0 |
| 2 | 3.6 | 1.7 | 1.3 | 1.2 | 2.5 | 2.1 | 2.1 | 0.916 |
| 4 | 16.9 | 15.6 | 14.8 | 16.8 | 15.9 | 16.1 | 16 | 0.771 |
| 8 | 41.1 | 38.4 | 38.7 | 41.6 | 41.4 | 39.9 | 40.2 | 1.4 |
| 12 | 63.1 | 62.4 | 63.4 | 64 | 64.5 | 62.6 | 63.4 | 0.809 |
| 16 | 82.4 | 83.1 | 83.7 | 84.9 | 82.8 | 81 | 83 | 1.297 |
| 20 | 98.1 | 98.1 | 99.4 | 100.4 | 101.7 | 98.2 | 99.3 | 1.504 |
| 24 | 98.1 | 99.2 | 100.7 | 101.1 | 103.6 | 103.4 | 101 | 2.211 |

### Example 3: Osmotic Pump Controlled Release Formulation C Comprising Nifedipine

The composition of the formulation C was as follows:

| | | mg/tablet | g/200 tablets | wt % |
|---|---|---|---|---|
| drug- | nifedipine | 99 | 19.8 | 13.20 |
| containing | polyoxyethylene | 328 | 65.6 | 43.73 |
| layer | N80 | | | |
| | sodium chloride | 47 | 9.4 | 6.27 |
| | magnesium stearate | 1 | 0.2 | 0.13 |
| push layer | polyoxyethylene | 213 | 42.6 | 28.40 |
| | WSR301 | | | |
| | sodium chloride | 60 | 12 | 8.00 |
| | iron oxide red | 1 | 0.2 | 0.13 |
| | magnesium stearate | 1 | 0.2 | 0.13 |

Manufacturer/log number information of active pharmaceutical ingredients and excipients:

Nifedipine: Moehs/17045001

Polyoxyethylene N80: Dow Chemical Company/14204001

Sodium chloride: AVANTOR MATERIALS/15077001

Magnesium stearate: MOLLINCKRODT/16347001

Polyoxyethylene WSR301: Dow Chemical Company/15296001

Iron oxide red: Huntsman Pigments and Additives/14196001

Cellulose acetate: Eastman Chemical Ltd./29360

Acetone: Guangzhou Chemical Reagent Factory/20171001-2

Equipment information:

Manual tablet press: CARVER/3850-7

High-efficiency coating machine: Zhejiang Xiaolun Pharmaceutical Machinery Co., Ltd/BGB-5F

Off-line automated sampling dissolution tester: SOTAX/AT xtend

High Performance Liquid Chromatography: Shimadzu/LC-20AT

The preparation process was as follows:

a) Preparation of the drug-containing layer mixture: Nifedipine, sodium chloride and polyoxyethylene N80 were mixed according to the composition in the above table. The mixture was sieved with a 20-mesh sieve to crush lumps. Finally, magnesium stearate was added and mixed homogeneously to obtain the mixture M. The mixture M was compressed into tablets with a punch having a diameter of 12 mm. The tablets were crushed in a mortar and sieved with a 20-mesh sieve to obtain the granular drug-containing layer mixture N.

b) Preparation of the push layer mixture: According to the above composition, polyoxyethylene WSR301, sodium chloride and iron oxide red were mixed homogeneously. Magnesium stearate was added into the mixture to obtain the powdery push layer mixture O.

c) Preparation of the core with channel: A punch with 12 mm diameter was used and there was a cylindrical protrusion on the surface of the punch. Firstly, the push layer mixture O was compressed to give a push layer. The drug-containing layer mixture N was compressed to give a drug-containing layer on the push layer and therefore a double-layered core with channel was obtained. The lateral length of the formed channel was 2.16 mm and the depth of the channel was 2.75 mm.

| | | Amount (g) |
|---|---|---|
| coating layer | cellulose acetate | 144 |
| | acetone | 4000 |

d) Film coating: Cellulose acetate was added into acetone. The mixture was stirred until cellulose acetate was dissolved to obtain a coating solution. The cores were placed in a coating machine and coated with the coating solution. The increase of coating weight was 5.33%.

The osmotic pump controlled release formulation C comprising nifedipine prepared according to the composition had one channel. The drug-containing layer of the core was communicated with the outer surface of the osmotic pump controlled release formulation C comprising nifedipine via the channel. The lateral length of the channel was 2.16 mm and the depth of the channel was 2.75 mm.

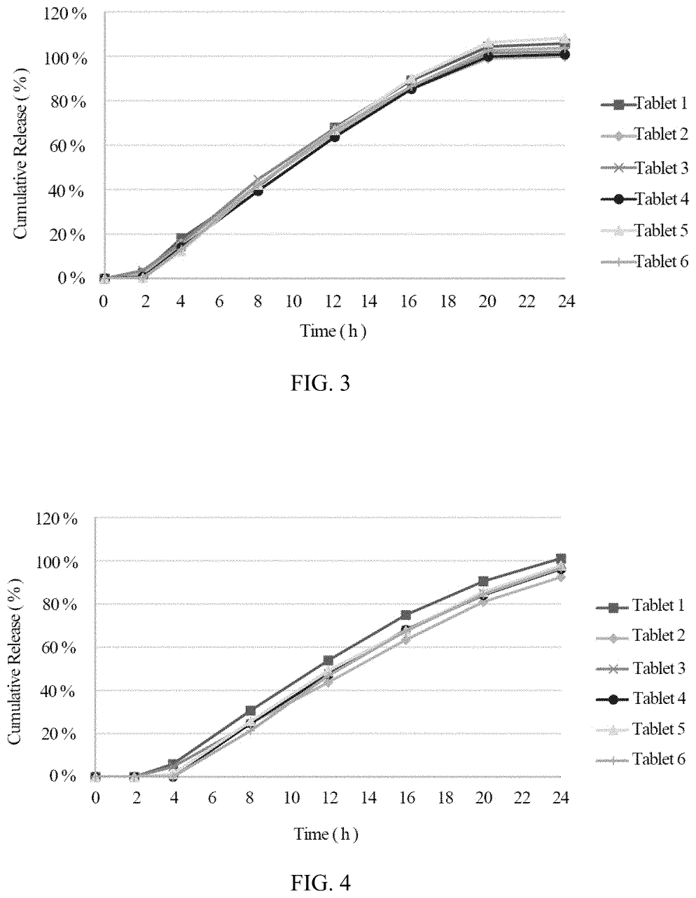

The dissolution of the osmotic pump controlled release formulation C comprising nifedipine was determined with the Apparatus II (Paddle) method specified in the United States Pharmacopoeia (USP), in which the speed of rotation was 100 rpm, the temperature of water bath was (37.0±0.5°) C., and the dissolution medium was 900 mL phosphate buffer solution of containing 1% sodium dodecyl sulfate (SLS) with pH=6.8. The dissolution test was carried out with dissolution tester and Agilent HPLC. 6 parallel samples (Tablets 1-6) of the osmotic pump controlled release formulation C comprising nifedipine were put into dissolution cups, respectively. 5 mL of solutions were sampled at 2 h, 4 h, 8 h, 12 h, 16 h, 20 h and 24 h, respectively. The samples were filtered with 0.45 μm microporous filter membrane, while 5 mL of dissolution medium with the same temperature was added. The subsequent filtrate was subject to HPLC. The cumulative release of the osmotic pump controlled release formulation C comprising nifedipine (Tablets 1-6) at different dissolution times were calculated, respectively, as shown in Table 3. The cumulative dissolution curves were plotted as shown in FIG. 3.

TABLE 3
Cumulative release of the osmotic pump controlled release formulation C comprising nifedipine (n = 6)

| Time | Cumulative Release (%) | | | | | | | |
|---|---|---|---|---|---|---|---|---|
| (h) | Tablet 1 | Tablet 2 | Tablet 3 | Tablet 4 | Tablet 5 | Tablet 6 | Mean | SD |
| 0 | 0 | 0 | 0 | 0 | 0 | 0 | 0 | 0 |
| 2 | 2.5 | 2.4 | 3.7 | 0.8 | 0.3 | 0.1 | 1.6 | 1.433 |
| 4 | 18.1 | 15.7 | 16 | 14.1 | 12.3 | 13.1 | 14.9 | 2.138 |
| 8 | 41.3 | 42.3 | 44.6 | 39.4 | 42.2 | 42.1 | 42 | 1.686 |
| 12 | 67.9 | 65.1 | 67.6 | 63.6 | 66.8 | 66.6 | 66.3 | 1.621 |
| 16 | 89.1 | 85.2 | 86.2 | 85.3 | 90 | 86.5 | 87.1 | 2.022 |
| 20 | 104.3 | 99 | 101.4 | 99.8 | 106.2 | 102.6 | 102.2 | 2.727 |
| 24 | 105.7 | 99.9 | 102 | 100.8 | 108.2 | 103.7 | 103.4 | 3.143 |

### Example 4: Osmotic Pump Controlled Release Formulation D Comprising Nifedipine

The composition of the formulation D was as follows:

| | | mg/tablet | g/200 tablets | wt % |
|---|---|---|---|---|
| drug- | nifedipine | 33 | 6.6 | 13.75 |
| containing | povidone K90 | 5 | 1 | 2.08 |
| layer | sodium chloride | 12 | 2.4 | 5.00 |
| | polyoxyethylene | 98.5 | 19.7 | 41.04 |
| | N80 | | | |
| | magnesium stearate | 1.5 | 0.3 | 0.63 |
| push layer | povidone K90 | 5 | 1 | 2.08 |
| | sodium chloride | 15.5 | 3.1 | 6.46 |
| | polyoxyethylene | 68.8 | 13.76 | 28.67 |
| | WSR301 | | | |
| | iron oxide red | 0.2 | 0.04 | 0.08 |
| | magnesium stearate | 0.5 | 0.1 | 0.21 |

Manufacturer/log number information of active pharmaceutical ingredients and excipients:

Nifedipine: Shanxi Xiyue Pharmaceutical Co., Ltd/A102718002

Povidone K90: BASF/96014768E0

Sodium chloride: Hebei Huachen Pharmaceutical Co., Ltd/1611001

Polyoxyethylene N80: Dow Chemical Company/E8123-17001

Magnesium stearate: Peter Greven Nederland C.V./C635909

Polyoxyethylene WSR301: Dow Chemical Company/WP436829

Iron oxide red: Shanghai Yipin Pigments Co., Ltd/20161007

Cellulose acetate: Eastman Chemical Ltd./29360

Acetone: Guangzhou Chemical Reagent Factory/20171001-2

Equipment information:

Manual tablet press: CARVER/3850-7

High efficiency coating machine: Zhejiang Xiaolun Pharmaceutical Machinery Co., Ltd/BGB-5F

Off-line automated sampling dissolution tester: SOTAX/AT xtend

High Performance Liquid Chromatography: Shimadzu/LC-20AT

The preparation process was as follows:

a) Preparation of the drug-containing layer mixture: Nifedipine, sodium chloride and polyoxyethylene N80 were mixed according to the composition in the above table. The mixture was sieved with a 20-mesh sieve to crush lumps and therefore the mixture P was obtained. Povidone K90 was added into purified water and stirred to prepare a 5% binder solution. The mixture P was put into a fluidized bed of type DPL-II. The binder solution was sprayed via top spray for granulation. Particulates were dried to obtain dry particles. Magnesium stearate was added into the dry particles and mixed to obtain the granular drug-containing layer mixture Q.

b) Preparation of the push layer mixture: According to the above composition, sodium chloride and polyoxyethylene WSR301 were mixed. The mixture was sieved with a 20-mesh sieve to crush lumps to obtain the mixture R. Povidone K90 was added into purified water and stirred to prepare a 5% binder solution. The mixture R was put into a fluidized bed of type DPL-II. The binder solution was sprayed via top spray for granulation. Particulates were dried to obtain dry particles. Magnesium stearate was added into the dry particles and mixed to obtain the granular drug-containing layer mixture S.

c) Preparation of the core with a channel: A punch with 8.70 mm diameter was used and there was a cylindrical protrusion on the surface of the punch. Firstly, the push layer mixture S was compressed to give a push layer. The drug-containing layer mixture Q was compressed to give a drug-containing layer on the push layer and therefore a double-layered core with channel was obtained. The lateral length of the formed channel was 2.00 mm and the depth of the channel was 2.50 mm.

| | | Amount (g) |
|---|---|---|
| coating layer | cellulose acetate | 144 |
| | acetone | 4000 |

d) Film coating: Cellulose acetate was added into acetone. The mixture was stirred until cellulose acetate was dissolved to obtain a coating solution. The cores were placed in a coating machine and coated with the coating solution. The increase of coating weight was 18.34%.

The osmotic pump controlled release formulation D comprising nifedipine prepared according to the composition had one channel. The drug-containing layer of the core was communicated with the outer surface of the osmotic pump controlled release formulation D comprising nifedipine via the channel. The lateral length of the channel was 2.00 mm and the depth of the channel was 2.50 mm.

The dissolution of the osmotic pump controlled release formulation D comprising nifedipine was determined with the Apparatus II (Paddle) method specified in the United States Pharmacopoeia (USP), in which the speed of rotation was 100 rpm, the temperature of water bath was (37.0±0.5°) C., and the dissolution medium was 900 mL phosphate buffer solution of containing 1% sodium dodecyl sulfate (SLS) with pH=6.8. The dissolution test was carried out with dissolution tester and Agilent HPLC. 6 parallel samples (Tablets 1-6) of the osmotic pump controlled release formulation D comprising nifedipine were put into dissolution cups, respectively. 5 mL of solutions were sampled at 2 h, 4 h, 8 h, 12 h, 16 h, 20 h and 24 h, respectively. The samples were filtered with 0.45 μm microporous filter membrane, while 5 mL of dissolution medium with the same temperature was added. The subsequent filtrate was subject to HPLC. The cumulative release of the osmotic pump controlled release formulation D comprising nifedipine (Tablets 1-6) at different dissolution times were calculated, respectively, as shown in Table 4. The cumulative dissolution curves were plotted as shown in FIG. 4.

TABLE 4
Cumulative release of the osmotic pump controlled release formulation D comprising nifedipine (n = 6)

| Time | Cumulative Release (%) | | | | | | | |
|---|---|---|---|---|---|---|---|---|
| (h) | Tablet 1 | Tablet 2 | Tablet 3 | Tablet 4 | Tablet 5 | Tablet 6 | Mean | SD |
| 0 | 0.00 | 0.00 | 0.00 | 0.00 | 0.00 | 0.00 | 0.00 | 0 |
| 2 | 0.00 | 0.00 | 0.00 | 0.00 | 0.00 | 0.00 | 0.00 | 0 |
| 4 | 5.90 | 0.00 | 4.70 | 0.00 | 1.20 | 0.00 | 2.00 | 0.026 |
| 8 | 30.70 | 24.80 | 24.50 | 24.80 | 25.70 | 21.30 | 25.30 | 0.03 |
| 12 | 53.80 | 43.70 | 47.70 | 47.30 | 49.20 | 46.80 | 48.10 | 0.034 |
| 16 | 74.90 | 63.30 | 67.90 | 68.00 | 67.60 | 67.40 | 68.20 | 0.037 |
| 20 | 90.50 | 81.00 | 85.00 | 84.10 | 85.50 | 84.50 | 85.10 | 0.031 |
| 24 | 101.10 | 92.40 | 97.00 | 96.10 | 97.70 | 96.60 | 96.80 | 0.028 |

### Example 5: Osmotic Pump Controlled Release Formulation E Comprising Nifedipine

The composition of the formulation E was as follows:

| | | mg/tablet | g/200 tablets | wt % |
|---|---|---|---|---|
| drug- | nifedipine | 33 | 6.6 | 13.75 |
| containing | povidone K90 | 5 | 1 | 2.08 |
| layer | sodium chloride | 12 | 2.4 | 5.00 |
| | polyoxyethylene | 98.5 | 19.7 | 41.04 |
| | N80 | | | |
| | magnesium stearate | 1.5 | 0.3 | 0.63 |
| push layer | povidone K90 | 5 | 1 | 2.08 |
| | sodium chloride | 15.5 | 3.1 | 6.46 |
| | polyoxyethylene | 68.8 | 13.76 | 28.67 |
| | WSR301 | | | |
| | iron oxide red | 0.2 | 0.04 | 0.08 |
| | Magnesium stearate | 0.5 | 0.1 | 0.21 |

Manufacturer/log number information of active pharmaceutical ingredients and excipients:

Nifedipine: Shanxi Xiyue Pharmaceutical Co., Ltd/A102718002

Povidone K90: BASF/96014768E0

Sodium chloride: Hebei Huachen Pharmaceutical Co., Ltd/1611001

Polyoxyethylene N80: Dow Chemical Company/E8123-17001

Magnesium stearate: Peter Greven Nederland C.V./c635909

Polyoxyethylene WSR301: Dow Chemical Company/WP436829

Iron oxide red: Shanghai Yipin Pigments Co., Ltd/20161007

Cellulose acetate: Eastman Chemical Ltd./29360

Acetone: Guangzhou Chemical Reagent Factory/20171001-2

Equipment information:

Manual tablet press: CARVER/3850-7

High-efficiency coating machine: Zhejiang Xiaolun Pharmaceutical Machinery Co., Ltd/BGB-5F

Off-line automated sampling dissolution tester: SOTAX/AT xtend

High Performance Liquid Chromatography: Shimadzu/LC-20AT

The preparation process was as follows:

a) Preparation of the drug-containing layer mixture: Nifedipine, sodium chloride and polyoxyethylene N80 were mixed according to the composition in the above table. The mixture was sieved with a 20-mesh sieve to crush lumps and therefore the mixture P was obtained. Povidone K90 was added into purified water and stirred to prepare a 5% binder solution. The mixture P was put into a fluidized bed of type DPL-II. The binder solution was sprayed via top spray for granulation. Particulates were dried to obtain dry particles. Magnesium stearate was added into the dry particles and mixed to obtain the granular drug-containing layer mixture Q.

b) Preparation of the push layer mixture: According to the above composition, sodium chloride and polyoxyethylene WSR301 were mixed. The mixture was sieved with a 20-mesh sieve to crush lumps to obtain the mixture R. Povidone K90 was added into purified water and stirred to prepare a 5% binder solution. The mixture R was put into a fluidized bed of type DPL-II. The binder solution was sprayed via top spray for granulation. Particulates were dried to obtain dry particles. Magnesium stearate was added into the dry particles and mixed to obtain the granular drug-containing layer mixture S.

c) Preparation of the core with a channel: A punch with 8.70 mm diameter was used and there was a cylindrical protrusion on the surface of the punch. Firstly, the push layer mixture S was compressed to give a push layer. The drug-containing layer mixture Q was compressed to give a drug-containing layer on the push layer and therefore a double-layered core with channel was obtained. The lateral length of the formed channel was 2.00 mm and the depth of the channel was 2.50 mm.

| | | Amount (g) |
|---|---|---|
| coating layer | cellulose acetate | 144 |
| | acetone | 4000 |

d) Film coating: Cellulose acetate was added into acetone. The mixture was stirred until cellulose acetate was dissolved to obtain a coating solution. The cores were placed in a coating machine and coated with the coating solution. The increase of coating weight was 16.58%.

The osmotic pump controlled release formulation E comprising nifedipine prepared according to the composition had one channel. The drug-containing layer of the core was communicated with the outer surface of the osmotic pump controlled release formulation E comprising nifedipine via the channel. The lateral length of the channel was 2.00 mm and the depth of the channel was 2.50 mm.

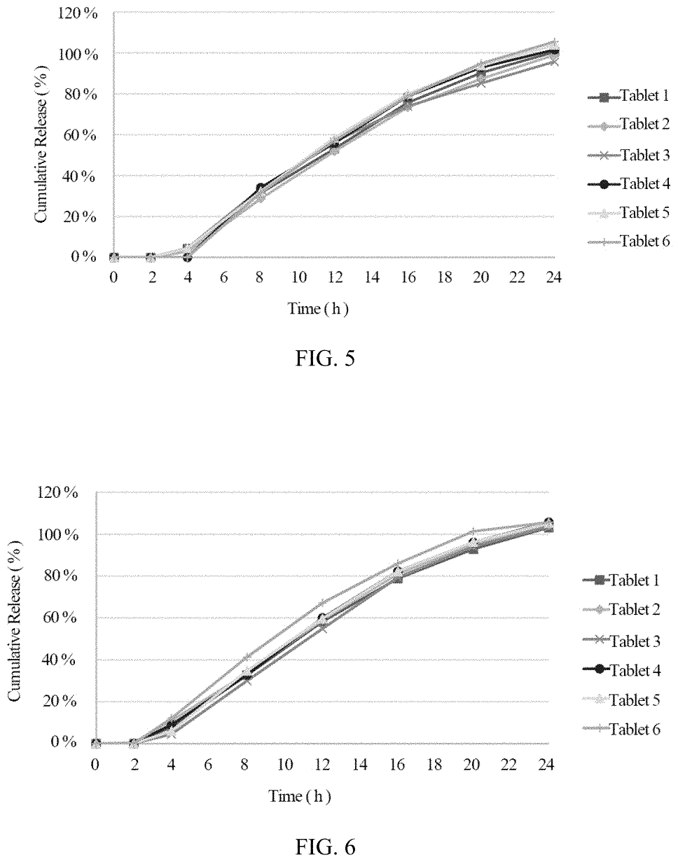

The dissolution of the osmotic pump controlled release formulation E comprising nifedipine was determined with the Apparatus II (Paddle) method specified in the United States Pharmacopoeia (USP), in which the speed of rotation was 100 rpm, the temperature of water bath was (37.0±0.5°) C., and the dissolution medium was 900 mL phosphate buffer solution of containing 1% sodium dodecyl sulfate (SLS) with pH=6.8. The dissolution test was carried out with dissolution tester and Agilent HPLC. 6 parallel samples (Tablets 1-6) of the osmotic pump controlled release formulation E comprising nifedipine were put into dissolution cups, respectively. 5 mL of solutions were sampled at 2 h, 4 h, 8 h, 12 h, 16 h, 20 h and 24 h, respectively. The samples were filtered with 0.45 μm microporous filter membrane, while 5 mL of dissolution medium with the same temperature was added. The subsequent filtrate was subject to HPLC. The cumulative release of the osmotic pump controlled release formulation E comprising nifedipine (Tablets 1-6) at different dissolution times were calculated, respectively, as shown in Table 5. The cumulative dissolution curves were plotted as shown in FIG. 5.

TABLE 5
Cumulative release of the osmotic pump controlled release formulation E comprising nifedipine (n = 6)

| Time | Cumulative Release (%) | | | | | | | |
|---|---|---|---|---|---|---|---|---|
| (h) | Tablet 1 | Tablet 2 | Tablet 3 | Tablet 4 | Tablet 5 | Tablet 6 | Mean | SD |
| 0 | 0.00 | 0.00 | 0.00 | 0.00 | 0.00 | 0.00 | 0.00 | 0 |
| 2 | 0.00 | 0.00 | 0.00 | 0.00 | 0.00 | 0.00 | 0.00 | 0 |
| 4 | 4.40 | 2.80 | 4.60 | 0.00 | 4.50 | 0.00 | 2.70 | 0.022 |
| 8 | 31.60 | 28.80 | 33.70 | 34.20 | 32.30 | 31.80 | 32.10 | 0.019 |
| 12 | 53.10 | 51.80 | 56.60 | 55.90 | 58.40 | 57.00 | 55.50 | 0.025 |
| 16 | 75.70 | 73.40 | 73.90 | 78.70 | 79.90 | 78.80 | 76.70 | 0.028 |
| 20 | 90.20 | 87.30 | 85.10 | 92.90 | 93.80 | 94.80 | 90.70 | 0.038 |
| 24 | 100.50 | 99.00 | 95.70 | 101.60 | 103.90 | 105.50 | 101.00 | 0.035 |

### Example 6: Osmotic Pump Controlled Release Formulation F Comprising Nifedipine

The composition of the formulation F was as follows:

| | | mg/tablet | g/200 tablets | wt % |
|---|---|---|---|---|
| drug- | nifedipine | 33 | 6.6 | 13.75 |
| containing | povidone K90 | 5 | 1 | 2.08 |
| layer | sodium chloride | 12 | 2.4 | 5.00 |
| | polyoxyethylene | 98.5 | 19.7 | 41.04 |
| | N80 | | | |
| | magnesium stearate | 1.5 | 0.3 | 0.63 |
| push layer | povidone K90 | 5 | 1 | 2.08 |
| | sodium chloride | 15.5 | 3.1 | 6.46 |
| | polyoxyethylene | 68.8 | 13.76 | 28.67 |
| | WSR301 | | | |
| | iron oxide red | 0.2 | 0.04 | 0.08 |
| | magnesium stearate | 0.5 | 0.1 | 0.21 |

Manufacturer/log number information of active pharmaceutical ingredients and excipients:

Nifedipine: Shanxi Xiyue Pharmaceutical Co., Ltd/A102718002

Povidone K90: BASF/96014768E0

Sodium chloride: Hebei Huachen Pharmaceutical Co., Ltd/1611001

Polyoxyethylene N80: Dow Chemical Company/E8123-17001

Magnesium stearate: Peter Greven Nederland C.V./C635909

Polyoxyethylene WSR301: Dow Chemical Company/WP436829

Iron oxide red: Shanghai Yipin Pigments Co., Ltd/20161007

Cellulose acetate: Eastman Chemical Ltd./29360

Acetone: Guangzhou Chemical Reagent Factory/20171001-2

Equipment information:

Manual tablet press: CARVER/3850-7

High-efficiency coating machine: Zhejiang Xiaolun Pharmaceutical Machinery Co., Ltd/BGB-5F

Off-line automated sampling dissolution tester: SOTAX/AT xtend

High Performance Liquid Chromatography: Shimadzu/LC-20AT

The preparation process was as follows:

a) Preparation of the drug-containing layer mixture: Nifedipine, sodium chloride and polyoxyethylene N80 were mixed according to the composition in the above table. The mixture was sieved with a 20-mesh sieve to crush lumps and therefore the mixture P was obtained. Povidone K90 was added into purified water and stirred to prepare a 5% binder solution. The mixture P was put into a fluidized bed of type DPL-II. The binder solution was sprayed via top spray for granulation. Particulates were dried to obtain dry particles. Magnesium stearate was added into the dry particles and mixed to obtain the granular drug-containing layer mixture Q.

b) Preparation of the push layer mixture: According to the above composition, sodium chloride and polyoxyethylene WSR301 were mixed. The mixture was sieved with a 20-mesh sieve to crush lumps to obtain the mixture R. Povidone K90 was added into purified water and stirred to prepare a 5% binder solution. The mixture R was put into a fluidized bed of type DPL-II. The binder solution was sprayed via top spray for granulation. Particulates were dried to obtain dry particles. Magnesium stearate was added into the dry particles and mixed to obtain the granular drug-containing layer mixture S.

c) Preparation of the core with a channel: A punch with 8.70 mm diameter was used and there was a cylindrical protrusion on the surface of the punch. Firstly, the push layer mixture S was compressed to give a push layer. The drug-containing layer mixture Q was compressed to give a drug-containing layer on the push layer and therefore a double-layered core with channel was obtained. The lateral length of the formed channel was 2.00 mm and the depth of the channel was 2.50 mm.

| | | Amount (g) |
|---|---|---|
| coating layer | cellulose acetate | 144 |
| | acetone | 4000 |

d) Film coating: Cellulose acetate was added into acetone. The mixture was stirred until cellulose acetate was dissolved to obtain a coating solution. The cores were placed in a coating machine and coated with the coating solution. The increase of coating weight was 14.21%.

The osmotic pump controlled release formulation F comprising nifedipine prepared according to the composition had one channel. The drug-containing layer of the core was communicated with the outer surface of the osmotic pump controlled release formulation F comprising nifedipine via the channel. The lateral length of the channel was 2.00 mm and the depth of the channel was 2.50 mm.

The dissolution of the osmotic pump controlled release formulation F comprising nifedipine was determined with the Apparatus II (Paddle) method specified in the United States Pharmacopoeia (USP), in which the speed of rotation was 100 rpm, the temperature of water bath was (37.0±0.5°) C., and the dissolution medium was 900 mL phosphate buffer solution of containing 1% sodium dodecyl sulfate (SLS) with pH=6.8. The dissolution test was carried out with dissolution tester and Agilent HPLC. 6 parallel samples (Tablets 1-6) of the osmotic pump controlled release formulation F comprising nifedipine were put into dissolution cups, respectively. 5 mL of solutions were sampled at 2 h, 4 h, 8 h, 12 h, 16 h, 20 h and 24 h, respectively. The samples were filtered with 0.45 μm microporous filter membrane, while 5 mL of dissolution medium with the same temperature was added. The subsequent filtrate was subject to HPLC. The cumulative release of the osmotic pump controlled release formulation F comprising nifedipine (Tablets 1-6) at different dissolution times were calculated, respectively, as shown in Table 6. The cumulative dissolution curves were plotted as shown in FIG. 6.

TABLE 6
Cumulative release of the osmotic pump controlled release formulation F comprising nifedipine (n = 6)

| Time | Cumulative Release (%) | | | | | | | |
|---|---|---|---|---|---|---|---|---|
| (h) | Tablet 1 | Tablet 2 | Tablet 3 | Tablet 4 | Tablet 5 | Tablet 6 | Mean | SD |
| 0 | 0.00 | 0.00 | 0.00 | 0.00 | 0.00 | 0.00 | 0.00 | 0 |
| 2 | 0.00 | 0.40 | 0.00 | 0.00 | 0.00 | 0.00 | 0.10 | 0.002 |
| 4 | 7.90 | 10.90 | 4.50 | 9.00 | 6.00 | 12.10 | 8.40 | 0.029 |
| 8 | 32.70 | 33.50 | 30.00 | 32.80 | 34.80 | 41.30 | 34.20 | 0.038 |
| 12 | 58.20 | 59.10 | 54.80 | 60.00 | 59.60 | 67.20 | 59.80 | 0.041 |
| 16 | 78.80 | 81.10 | 79.30 | 82.30 | 82.20 | 86.00 | 81.60 | 0.026 |
| 20 | 92.80 | 95.10 | 94.10 | 96.10 | 96.20 | 101.40 | 95.90 | 0.029 |
| 24 | 103.10 | 104.40 | 103.60 | 105.70 | 105.30 | 105.70 | 104.60 | 0.011 |

### Example 7: Osmotic Pump Controlled Release Formulation G Comprising Carbamazepine

The composition of the formulation G was as follows:

| | | mg/tablet | g/200 tablets | wt % |
|---|---|---|---|---|
| drug- | carbamazepine | 400 | 160 | 58.82 |
| containing | hydroxypropyl methyl | 30 | 12 | 4.41 |
| layer | cellulose E3 | | | |
| | 5% hydroxypropyl | 10 | 4 | 1.47 |
| | methyl cellulose | | | |
| | E3 solution | | | |
| | hydroxyethyl cellulose | 77 | 30.8 | 11.32 |
| | sodium chloride | 150 | 60 | 22.06 |
| | sodium dodecyl sulfate | 5 | 2 | 0.74 |
| | iron oxide yellow | 0.85 | 0.34 | 0.13 |
| | iron oxide red | 0.15 | 0.06 | 0.02 |
| | magnesium stearate | 7 | 2.8 | 1.03 |

Manufacturer/log number information of active pharmaceutical ingredients and excipients:

Carbamazepine: Zhejiang Jiuzhou Pharmaceutical Co., Ltd./CN160914101

Hypromellose E3: Dow Chemical Company/PDR453814

Hydroxyethyl cellulose: Ashland/M1706

Sodium chloride: Hebei Huachen Pharmaceutical Co., Ltd/161221

Sodium dodecyl sulfate: BASF/0015079802

Iron Oxide Yellow: Sen Xiang Jing Pigment Technology (China) Co., Ltd/5309052

Iron oxide red: Shanghai Yipin Pigments Co., Ltd/20161007

Magnesium stearate: Peter Greven Nederland C.V./C635909

PEG 6000: Dow Chemical Company/D684H82BG1

Cellulose acetate: Eastman Chemical Ltd./29360

Acetone: Guangzhou Chemical Reagent Factory/20171001-2

Equipment information:

Manual tablet press: CARVER/3850-7

High-efficiency coating machine: Zhejiang Xiaolun Pharmaceutical Machinery Co., Ltd/BGB-5F

Off-line automated sampling dissolution tester: SOTAX/AT xtend

Ultraviolet visible spectrophotometer: Shimadzu/UV-2600

The preparation process was as follows:

a) Preparation of the drug-containing layer mixture: Hydroxypropyl methyl cellulose E3, hydroxyethyl cellulose, sodium chloride, sodium dodecyl sulfate, iron oxide yellow and iron oxide red were mixed according to the composition in the above table. The mixture was sieved with a 20-mesh sieve to crush lumps. Magnesium stearate was added into the mixture and mixed to obtain the drug-containing layer mixture T.

b) Preparation of the core with a channel: A punch with 11.90 mm diameter was used and there was a cylindrical protrusion on the surface of the punch. The drug-containing layer mixture T was compressed to give a single-layered core with channel. The lateral length of the formed channel was 2.00 mm and the depth of the channel was 2.50 mm.

| | | Amount (g) |
|---|---|---|
| coating layer | PEG 6000 | 112.55 |
| | purified water | 855.15 |
| | cellulose acetate | 262.56 |
| | acetone | 6270 |

c) Film coating: PEG 6000 was added into purified water. The mixture was stirred until PEG 6000 was dissolved to obtain a PEG solution. The cellulose acetate was added into acetone. The mixture was stirred until cellulose acetate was dissolved. The PEG solution was added into the cellulose acetate solution. The resultant mixture was stirred to obtain a coating solution. The cores were placed in a coating machine and coated with the coating solution. The increase of coating weight was 3.00%.

The osmotic pump controlled release formulation G comprising carbamazepine prepared according to the composition had one channel. The drug-containing layer of the core was communicated with the outer surface of the osmotic pump controlled release formulation G comprising carbamazepine via the channel. The lateral length of the channel was 2.00 mm and the depth of the channel was 2.50 mm.

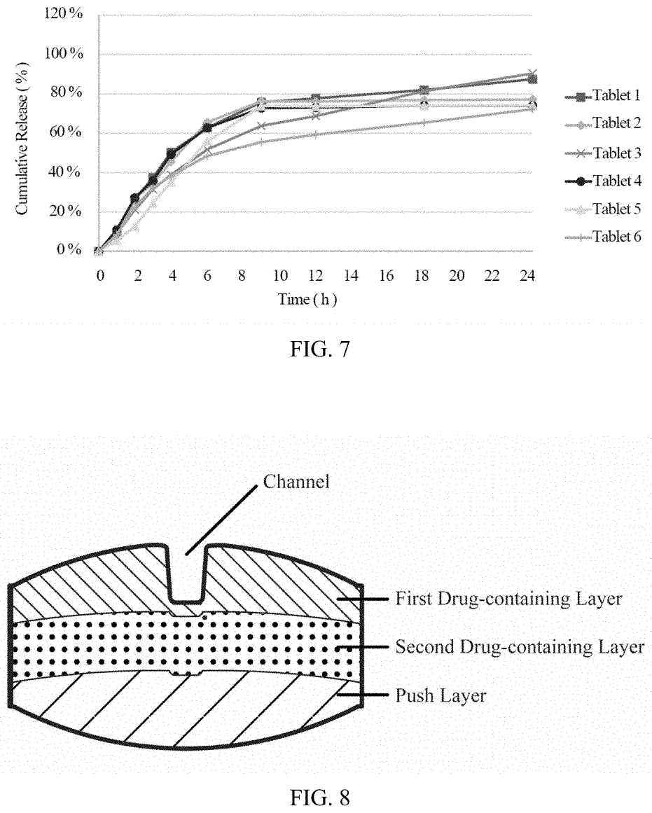

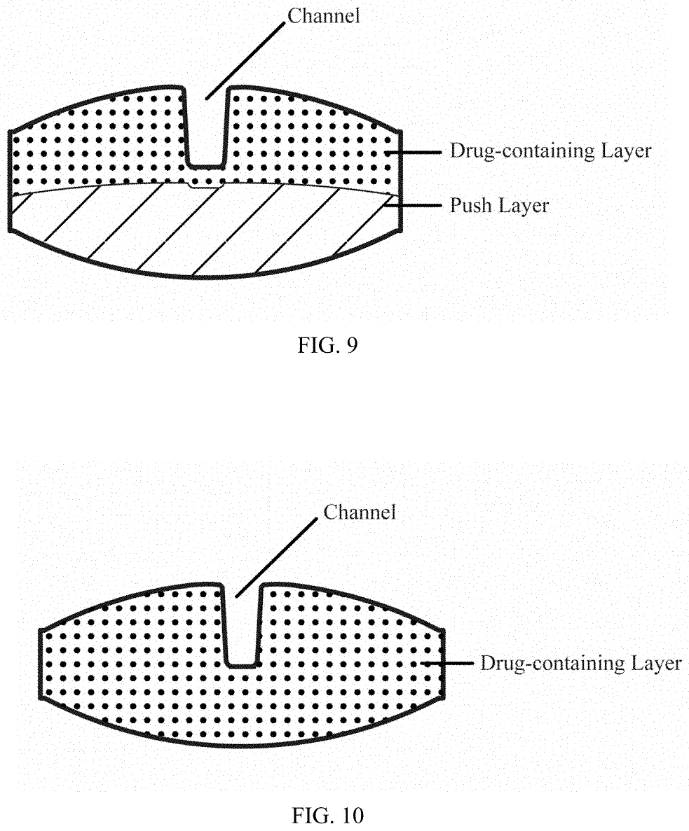

The dissolution of the osmotic pump controlled release formulation G comprising carbamazepine was determined with the Apparatus II (Paddle) method specified in the United States Pharmacopoeia (USP), in which the speed of rotation was 100 rpm, the temperature of water bath was (37.0±0.5°) C., and the dissolution medium was 900 mL phosphate buffer solution containing 1% sodium dodecyl sulfate (SLS) with pH=6.8. The dissolution test was carried out with dissolution tester and Agilent HPLC. 6 parallel samples (Tablets 1-6) of the osmotic pump controlled release formulation G comprising carbamazepine were put into dissolution cups, respectively. 5 mL of solutions were sampled at 2 h, 4 h, 8 h, 12 h, 16 h, 20 h and 24 h, respectively. The samples were filtered with 0.45 μm microporous filter membrane, while 5 mL of dissolution medium with the same temperature was added. The subsequent filtrate was subject to HPLC. The cumulative release of the osmotic pump controlled release formulation G comprising carbamazepine (Tablets 1-6) at different dissolution times were calculated, respectively, as shown in Table 7. The cumulative dissolution curves were plotted as shown in FIG. 7.

TABLE 7
Cumulative release of the osmotic pump controlled release formulation G comprising carbamazepine (n = 6)

| Time | Cumulative Release (%) | | | | | | | |
|---|---|---|---|---|---|---|---|---|
| (h) | Tablet 1 | Tablet 2 | Tablet 3 | Tablet 4 | Tablet 5 | Tablet 6 | Mean | SD |
| 0 | 0.00 | 0.00 | 0.00 | 0.00 | 0.00 | 0.00 | 0.00 | 0 |
| 1 | 8.40 | 9.50 | 8.90 | 11.00 | 5.60 | 8.50 | 8.60 | 0.018 |
| 2 | 26.50 | 26.60 | 21.20 | 27.20 | 12.60 | 22.40 | 22.80 | 0.055 |
| 3 | 37.40 | 34.80 | 31.50 | 35.90 | 24.60 | 32.30 | 32.70 | 0.046 |
| 4 | 50.30 | 45.50 | 38.90 | 49.20 | 35.20 | 38.40 | 42.90 | 0.063 |
| 6 | 62.80 | 65.60 | 51.70 | 62.70 | 55.80 | 48.40 | 57.80 | 0.069 |
| 9 | 75.80 | 76.20 | 63.80 | 72.80 | 73.90 | 55.60 | 69.70 | 0.083 |
| 12 | 77.70 | 76.20 | 68.80 | 73.10 | 73.70 | 59.30 | 71.50 | 0.067 |
| 18 | 82.00 | 76.90 | 81.40 | 74.10 | 74.00 | 65.40 | 75.60 | 0.061 |
| 24 | 87.50 | 77.20 | 90.40 | 74.10 | 74.20 | 72.30 | 79.30 | 0.077 |

As used herein, relational terms, such as first, second and the like, are merely used to distinguish one entity or operation from another entity or operation, but do not necessarily require or imply any such actual relationship or order among these entities or operations.

From the foregoing, it would be appreciated that although specific embodiments of the present disclosure have been described for illustrative purposes, various modifications or improvements can be made by those skilled in the art without departing from the spirit and scope of the present disclosure. Such variations or modifications are intended to fall within the scope of the attached claims of the present disclosure.

What is claimed is:

1. An osmotic pump controlled release formulation, comprising a core and a membrane, wherein the membrane is coated on an outer surface of the core, the formulation has at least one channel, the core is communicated with the outer surface of the formulation via the channel, lateral length of the channel is 1.40 mm-2.50 mm, and depth of the channel is 2.05 mm-2.95 mm.

2. The osmotic pump controlled release formulation of claim 1, wherein the lateral length of the channel is 1.70 mm-2.20 mm, and the depth of the channel is 2.25 mm-2.75 mm.

3. The osmotic pump controlled release formulation of claim 1, wherein bottom shape of the channel is circular, oval, square, rectangular, diamond or triangular.

4. The osmotic pump controlled release formulation of claim 1, wherein the core is single-layered or multi-layered.

5. The osmotic pump controlled release formulation of claim 1, wherein the core comprises at least one drug-containing layer.

6. The osmotic pump controlled release formulation of claim 1, wherein the core is double-layered.

7. The osmotic pump controlled release formulation of claim 6, wherein a first layer of the double layers is a drug-containing layer, and a second layer of the double layers is a push layer.

8. The osmotic pump controlled release formulation of claim 1, wherein the core is three-layered.

9. The osmotic pump controlled release formulation of claim 8, wherein a first layer of the three layers is a drug-containing layer, a second layer of the three layers is a drug-containing layer, and a third layer of the three layers is a push layer.

10. The osmotic pump controlled release formulation of claim 1, wherein the core comprises an active pharmaceutical ingredient and the active pharmaceutical ingredient is selected from the group consisting of antihypertensive agents, hypolipidemic agents, oral anti-diabetic agents, non-steroidal anti-inflammatory agents, β-receptor antagonists, calcium channel blockers, serotonin reuptake inhibitors, antipsychotic agents and antibacterial agents.

11. The osmotic pump controlled release formulation of claim 10, wherein the active pharmaceutical ingredient is selected from the group consisting of prazosin hydrochloride, phenylpropanolamine hydrochloride, doxazosin mesylate, verapamil hydrochloride, oxybutynin hydrochloride, isradipine, paliperidone, chlorpheniramine maleate, glipizide, nifedipine, pseudoephedrine hydrochloride, pseudoephedrine sulfate, aluminum sulfate, carbamazepine, leuprorelin acetate, sufentanil citrate and methylphenidate hydrochloride.

12. The osmotic pump controlled release formulation of claim 1, wherein the membrane comprises a semi-permeable membrane material.

13. The osmotic pump controlled release formulation of claim 12, wherein the semi-permeable membrane material is selected from the group consisting of cellulose acetate, aqueous cellulose acetate dispersion, ethyl cellulose, polyvinyl chloride, polycarbonate, vinyl alcohol-vinyl acetate, ethylene-propylene polymer and a mixture thereof.

14. The osmotic pump controlled release formulation of claim 1, wherein the membrane further comprises a pore-forming agent.

15. The osmotic pump controlled release formulation of claim 14, wherein the pore-forming agent is selected from the group consisting of glycerol, sorbitol, polyethylene glycol 400, polyethylene glycol 600, polyethylene glycol 1000, polyethylene glycol 1500, polyethylene glycol 4000, hydroxypropyl methylcellulose, polyvinyl alcohol and a mixture thereof.

16. A process for preparing an osmotic pump controlled release formulation, comprising:
compressing a core and forming a channel during the compression to obtain a core with at least one channel, and
coating the compressed core to obtain the osmotic pump controlled release formulation,
wherein lateral length of the channel is 1.40 mm-2.50 mm and depth of the channel is 2.05 mm-2.95 mm.

17. The process of claim 16, wherein an increase of coating weight is 1%-20%.

18. A method for improving drug release in a subject, comprising administering an osmotic pump controlled release formulation to an subject in need thereof, wherein the osmotic pump controlled release formulation comprises a core and a membrane, the membrane is coated on an outer surface of the core, the formulation has at least one channel, the core is communicated with the outer surface of the formulation via the channel, lateral length of the channel is 1.40 mm-2.50 mm, and depth of the channel is 2.05 mm-2.95 mm.
